(19) United States
(12) Patent Application Publication
(10) Pub. No.: US 2015/0134348 A1
(43) Pub. Date: May 14, 2015

(54) METHOD AND SYSTEM FOR SELECTING THE FREQUENCY OF ARTERIAL BLOOD GAS TESTING FOR NEONATES

(71) Applicant: KONINKLIJKE PHILIPS N.V., EINDHOVEN (NL)

(72) Inventor: Vishnu Vardhan Makkapati, Ongole (IN)

(73) Assignee: Koninklijke Philips N.V., Eindhoven (NL)

(21) Appl. No.: 14/401,148

(22) PCT Filed: May 14, 2013
(86) PCT No.: PCT/IB2013/053915

§ 371 Date: Nov. 14, 2014

Related U.S. Application Data
(60) Provisional application No. 61/654,224, filed on Jun. 1, 2012.

(51) Int. Cl. G06F 19/00; A61B 5/1455

(52) U.S. Cl. CPC G06F 19/327; G06F 19/321; G06F 19/363; A61B 5/14551
USPC 705/2

(57) ABSTRACT

A system and method for receiving previous arterial blood gas (“ABG”) test results for a patient, determining an initial time for a next ABG test for the patient based on the previous ABG test results, receiving monitoring data for the patient and determining a modified time for a next ABG test based on the initial time for the next ABG test and the patient monitoring data.

Neonates suffering from severe respiratory stress are typically put on a ventilator and are regularly monitored to assess changes in their clinical condition. The settings of the ventilator may be changed depending on how the neonate is responding to treatment, with the response evaluated by measuring various parameters. The Arterial Blood Gas (“ABG”) test is an important test that is conducted to measure key parameters for adjusting ventilator settings. However, the ABG test is both expensive to conduct and painful to administer; therefore, it is desirable to optimize the selection of the frequency at which it is performed.

A method for receiving previous arterial blood gas (“ABG”) test results for a patient, determining an initial time for a next ABG test for the patient based on the previous ABG test results, receiving monitoring data for the patient and determining a modified time for a next ABG test based on the initial time for the next ABG test and the patient monitoring data.

A system having a patient monitor detecting monitoring data for a patient, a memory storing previous arterial blood gas (“ABG”) test results for the patient and an initial time for a next ABG test determined based on the previous ABG test results and a processor determining a modified time for a next ABG test based on the initial time for the next ABG test and the patient monitoring data.

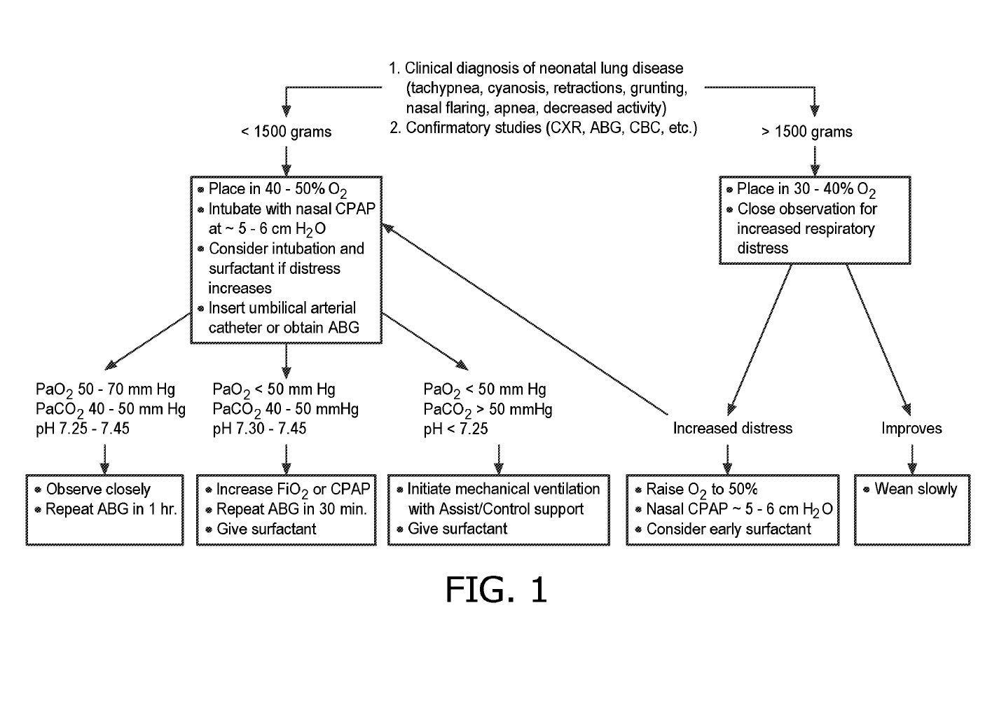

FIG. 1 shows an exemplary set of rules governing assisted ventilation of a neonate.

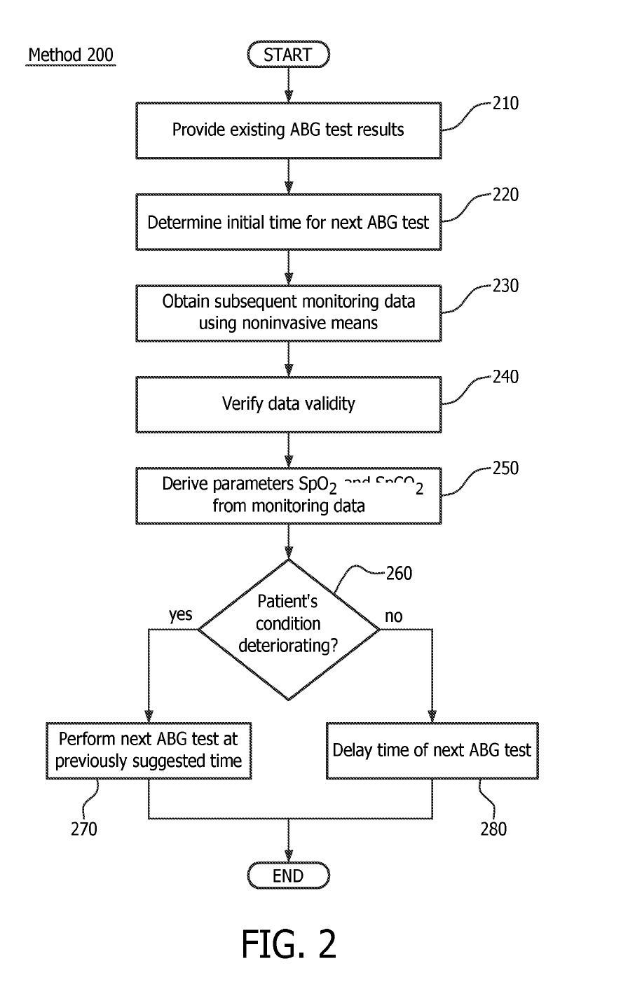

FIG. 2 shows an exemplary method for selecting a frequency of ABG testing according to an exemplary embodiment.

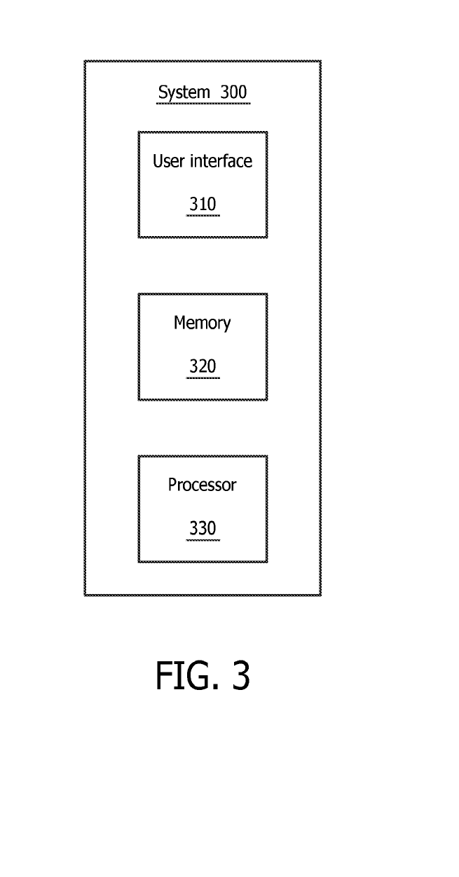

FIG. 3 shows an exemplary system for selecting a frequency of ABG testing according to an exemplary embodiment.

The exemplary embodiments may be further understood with reference to the following description of exemplary embodiments and the related appended drawings, wherein like elements are provided with the same reference numerals. Specifically, the exemplary embodiments relate to methods and systems for optimizing the selection of the frequency of arterial blood gas (“ABG”) testing for neonatal intensive care patients.

Neonates (i.e., newborns) who are being treated in a neonatal intensive care unit (“NICU”) for severe respiratory distress are typically treated with a ventilator and are continuously monitored using patient monitors, ventilator parameters, and various other tests. The results of this monitoring is parameters that are used to adjust the parameters of the ventilator. One important test is the ABG test, which is used to obtain values for partial pressure of oxygen (“PaO2”) and partial pressure of carbon dioxide (“PaCO2”). However, the ABG test is both invasive, and therefore painful to the neonate, and expensive to administer. Therefore, it is highly desirable to perform ABG testing only at optimal time intervals, in order to minimize both the infliction of pain on the neonate and the cost of the testing.

Typically, based on the results of the most recent ABG test, the settings of the ventilator may be adjusted and the time for the next ABG test may be selected. FIG. 1 illustrates an exemplary set of rules governing such selections, as defined in “Assisted Ventilation of the Neonate”, by Jay P. Goldsmith and Edward H. Karotkin, Fifth Edition, 2010. It will be apparent to those of skill in the art that this set of rules defines the subsequent treatment of the neonate, including adjusting the ventilator settings (or leaving the settings unchanged) and determining a time to repeat the ABG test.

FIG. 2 illustrates an exemplary method 200 for optimizing the determination of the time to conduct a subsequent ABG test. In step 210, the results of an existing ABG test are provided. This may entail the consideration of the results of a current ABG test or, alternately, the retrieval of the results of a previously-performed test, such as from a medical records database or any other suitable storage medium. In step 220, an initial time for a next ABG test is determined based on the results of the existing ABG test using known methods, such as the methodology outlined in FIG. 1.

In step 230, subsequent monitoring data for the neonatal patient is obtained by noninvasive means. This step may include testing blood oxygen saturation (“SpO2”) using a pulse oximeter and testing for end-tidal carbon dioxide (“EtCO2”) using the ventilator or other another capnographic technique. These values may also be obtained using transcutaneous monitoring (e.g., tcO2 and tcCO2) or an SpO2 camera. The patient may also be monitored using a camera (e.g., an analog or digital video camera or camera capturing a series of still images), which may detect changes in the skin tone of the patient.

In step 240, the validity of the data obtained in step 230 is verified. This step may be necessary because the data may not be reliable under certain conditions (e.g., depending on the type of monitoring used to obtain the data, or on the patient's condition, such as apnea of prematurity). For example, if SpO2 is one of the types of patient monitoring data obtained in step 230, tracings of SpO2 may be used to determine the validity. Alternately, a reliable value for SpO2 may be obtained using Signal Extraction Technology.

In step 250, the parameters PaO2 and PaCO2 are derived from the patient data obtained in step 230 and validated in step 240. Those of skill in the art will understand that there are various means for performing such derivation. In one exemplary embodiment, PaO2 may be determined based on SpO2 using the expression:

PaO2=(0.03)·e^{0.08(SpO2)}

In the above expression, PaO2 is expressed in Torr. Additionally, PaCO2 may be determined based on EtCO2, as described in “Relationship Between Arterial Carbon Dioxide And End-Tidal Carbon Dioxide When A Nasal Sampling Port Is Used”, by Stephen E. McNulty et al., Journal of Clinical Monitoring, April 1990.

In step 260, it is determined whether the patient's condition is deteriorating. This determination may be made by comparing the derived values of PaO2 and PaCO2 to their values as measured during the most recent ABG test, and by comparing them to prescribed ranges (e.g., as illustrated in FIG. 1). The determination of the patient's condition may also involve the examination of camera-recorded still or video imagery; for example, neonates may become pale when oxygenation is insufficient. Alternately, the patient can be visually observed by a neonatologist, who may then indicate whether the patient has become pale.

If the patient's condition is determined to be deteriorating in step 260, then, in step 270, the next ABG test is performed at or within the time suggested by the results of the previous ABG test. In contrast, if, in step 260, the patient's condition is determined not to be deteriorating, then, in step 280, the time of the next ABG test is delayed to the time that may be suggested by the method illustrated in FIG. 1. After either step 270 or step 280, the method 200 terminates. However, those of skill in the art will understand that the input measurements described above may be obtained continually and noninvasively, and so the method 200 may be continuously performed between ABG tests in order to provide up-to-date monitoring of the patient's condition In one embodiment, the determination of whether the patient's condition is deteriorating, and the resulting recommendation of the time for the next ABG test, may be made by a clinical decision support system, as described hereinafter.

FIG. 3 illustrates an exemplary system 300 for determining an optimal time for a next ABG test using a method such as the method 200. The system 300 includes a user interface 310, which may receive input data regarding ABG tests and other patient monitoring data as described above. In one embodiment, the user interface may be coupled directly to patient monitoring information in order to simplify the data communication process. The system 300 additionally includes a memory 320 storing a program embodying a method such as the method 200, and a processor 330 performing the method in order to provide output as described above. The output may be provided by means of the user interface 310.

The exemplary embodiments enable the timing of the ABG test to be optimized. As described above, this may be accomplished automatically using a system such as a clinical decision support system that may receive input from a clinician and output a recommended time. As a result, the costs of administering a series of ABG tests may be minimized, and neonatal patients may be spared from more invasive procedures than are necessary.

It is noted that the claims may include reference signs/numerals in accordance with PCT Rule 6.2(b). However, the present claims should not be considered to be limited to the exemplary embodiments corresponding to the reference signs/numerals.

It will be apparent to those skilled in the art that various modifications may be made to the exemplary embodiments, without departing from the spirit or the scope of the invention. Thus, it is intended that the present invention cover modifications and variations of this invention provided they come within the scope of the appended claims and their equivalents.

1. A method, comprising:
receiving previous arterial blood gas (“ABG”) test results for a patient;
determining an initial time for a next ABG test for the patient based on the previous ABG test results and a set of rules defining subsequent treatment of the patient;
receiving monitoring data for the patient;
determining a modified time for a next ABG test based on the initial time for the next ABG test and the patient monitoring data; and
deriving patient oxygenation parameters based on the monitoring data,
wherein the modified time for the next ABG test is determined based on the initial time for the next ABG test and the patient oxygenation parameters;
wherein determining the modified time for the next ABG test comprises delaying the next ABG test if the monitoring data indicates that a condition of the patient has not deteriorated; and
wherein determining the modified time for the next ABG test comprises accelerating the next ABG test if the monitoring data indicates that a condition of the patient has deteriorated.

2. (canceled)

3. The method of claim 1, wherein the patient oxygenation parameters are PaO2 and PaCO2.

4. The method of claim 3, wherein PaO2 is determined based on one of SpO2 and tcCO2.

5. The method of claim 4, wherein SpO2 is determined based on the results of a pulse oximeter test.

6. The method of claim 3, wherein PaCO2 is determined based on one of EtCO2 and tcCO2.

7. (canceled)

8. (canceled)

9. The method of claim 1, further comprising:
receiving an image of the patient,
wherein the modified time for the next ABG test is further determined based on the image of the patient.

10. The method of claim 9, wherein the determination is based on whether the image indicates that the patient is paler than a previous image.

11. A system, comprising:
a patient monitor detecting monitoring data for a patient;
a memory storing previous arterial blood gas (“ABG”) test results for the patient and an initial time for a next ABG test determined based on the previous ABG test results; and
a processor determining a modified time for a next ABG test based on the initial time for the next ABG test and the patient monitoring data;
wherein the processor further derives patient oxygenation parameters based on the monitoring data, wherein the modified time for the next ABG test is determined based on the initial time for the next ABG test and the patient oxygenation parameters;
wherein the processor determines the modified time for the next ABG test comprises delaying the next ABG test if the monitoring data indicates that a condition of the patient has not deteriorated; and
wherein the processor determines the modified time for the next ABG test comprises accelerating the next ABG test if the monitoring data indicates that a condition of the patient has deteriorated.

12. (canceled)

13. The system of claim 1, wherein the patient oxygenation parameters are PaO2 and PaCO2.

14. The system of claim 13, wherein PaO2 is determined based on one of SpO2 and tcCO2.

15. The system of claim 14, wherein SpO2 is determined based on the results of a pulse oximeter test.

16. The system of claim 13, wherein PaCO2 is determined based on one of EtCO2 and tcCO2.

17. (canceled)

18. (canceled)

19. The system of claim 11, wherein the processor receives an image of the patient, wherein the modified time for the next ABG test is further determined based on the image of the patient.

20. The system of claim 19, wherein the determination is based on whether the image indicates that the patient is paler than a previous image.
